(19) United States
(12) Patent Application Publication
(10) Pub. No.: US 2002/0155614 A1
(43) Pub. Date: Oct. 24, 2002

(54) Peptide esterification

(76) Inventors: Andrew J. Tomlinson, Wayland, MA (US); Marina Hincapie, Framingham, MA (US); Roman M. Chicz, Belmont, MA (US)

Correspondence Address: JANIS K. FRASER, PH.D, Fish & Richardson P.C., 225 Franklin Street, Boston, MA, 02110-2804, US

(21) Appl. No.: 10/081,379

(22) Filed: Feb. 21, 2002

Related U.S. Application Data
(60) Provisional application No. 60/270,336, filed on Feb. 21, 2001.
(60) Provisional application No. 60/284,416, filed on Apr. 16, 2001.

(51) Int. Cl. G01N 33/00; C07K 7/00

(52) U.S. Cl. 436/86; 530/345

(57) ABSTRACT

The invention provides methods and reagents for the esterification of peptides. The methods can be used to (i) enable a peptide in a first sample to be quantified relative to the level of the same peptide in a second sample, (ii) identify peptides in a complex mixtures that are targets for sequencing efforts, and (iii) aid peptide sequence elucidation. Methods that promote esterification and the formation of esters that are labeled with stable isotopes are described. These methods are useful for the analysis of low concentration (e.g., sub-femtomole) peptide mixtures.

### CROSS REFERENCE TO RELATED APPLICATIONS

This application claims priority from U.S. Provisional Application No. 60/270,336, filed Feb. 21, 2001, and U.S. Provisional Application No. 60/284,416, filed Apr. 16, 2001. These applications are incorporated herein by reference in their entirety.

### FIELD OF THE INVENTION

This invention relates generally to peptides, and in particular to methods of esterifying peptides.

### BACKGROUND OF THE INVENTION

Methods of detecting and quantitating mRNA levels are widely used in approaches for profiling gene expression. Proteome analysis, the analysis of protein expression, is a complementary method for the study of gene expression. As compared to the study of gene expression at the mRNA level, proteome analysis provides detailed information about biological systems by focusing directly on the proteins, rather than on the nucleic acids that encode them.

Isotopic labeling of peptides constitutes one mechanism that can be used for the quantification and sequence identification of individual proteins within complex mixtures (see, e.g., Gygi et al. (1999) Nature Biotechnology 17:994-999 and WO 00/11208). For example, Gygi et al. described the synthesis of hydrogen- and deuterium-labeled isotope-coded affinity tag (ICAT) reagents that are used to alkylate cysteine residues of peptides. ICAT reagents also contain biotin functionality to permit the use of an avidin column to specifically isolate derivatized peptides, thereby simplifying the peptide mixture for analysis and enabling the detection of proteins that are expressed at low levels. Cysteine-containing peptides constitute only a fraction of the peptides in some complex protein mixtures.

### SUMMARY OF THE INVENTION

The methods described herein involve the esterification of a peptide species or a population of peptides. According to these methods, peptides that contain an acidic residue or free acid C-terminus can be derivatized. In addition, peptides can optionally be isotopically labeled. The peptide esterification methods can be used, for example, to determine relative protein quantities and can aid in sequence identification of proteins in complex mixtures.

In one aspect, the invention features a method for preparing a peptide ester. The method includes the steps of: providing an acidified alcohol solution; providing a peptide sample comprising a peptide species; and mixing the acidified alcohol solution and the peptide sample to form a mixture and thereby generate an ester of the peptide species, wherein concentration of the peptide species in the mixture is less than 1 nM.

The method for preparing a peptide ester can further include a step of adsorption of the peptide species onto a solid phase prior to the mixing step. Example solid phases include a hydrophilic chromatography phase and a strong cation exchanger.

In one example, the ester is a methyl ester.

The method can optionally include a step of detecting the peptide species. For example, the method can further include sequencing the peptide species after generation of the ester.

In one embodiment, the method features microchemistry that yields quantitative methylation of low concentrations (e.g., pM to nM levels) of a peptide or peptides. “Microchemistry” refers to a scale of chemistry that permits efficient derivatization of a peptide in a sample of a complex mixture in which the peptide sample, or an individual peptide species, is at a concentration of less than 1 nM. A complex mixture of peptides includes one that has been derived from a cell or a tissue.

As used herein, a “peptide ester” refers to a peptide that has been chemically modified through one or more acidic free residues or free acid C-terminus. The term “peptide” is used synonymously with “protein” and “polypeptide.” In one example, peptides used herein are 2-30 amino acids in length, e.g., naturally processed HLA-binding peptides. Peptides may be supplied in a variety of forms, e.g., dry such as lyophilized, or in a mixture, e.g., reconstituted in solution. Alcohols useful in preparing peptide esters of the invention include methanol, ethanol, propanol, and isopropanol.

Additionally, the alcohols used in performing the methods of the invention may be substituted with specific functional groups, which effectively change the chemical properties of esterified peptides that are generated by this reaction. Examples of useful substituted alcohols include aminoethanol, trialkyl ammonium ethanol, biotinylated alcohol, and histidine labeled alcohol.

According to the methods describe herein, peptides may further be adsorbed onto a solid phase. Techniques for adsorbing peptides onto solid phase are well known in the art and include the use of hydrophilic chromatography (HILIC) phase mechanisms, which use mobile phases (such as an acetonitrile to water gradient), salt or pH gradient to recover peptide esters after they are produced. Ion exchange solid phases (e.g., strong or weak cation exchangers and strong or weak anion exchangers) using a salt or pH gradient to elute the peptide esters can also be used for this application. Reversed phase resins such as C2, C4, C8, and C18 or the polymeric reversed phase solid phases (e.g., Poros R1, R2, and/or R3, and PLRP-S type phases) can also be used to adsorb peptides. The term, “solid phase”, as used herein, is meant to denote any stationary support or matrix system to which the peptides can be adsorbed. For the purposes of this application, the terms “solid phase” and “stationary phase” are used interchangeably.

Examples of solid phases that are useful include HILIC (a strong cation exchanger that can use sulfonic acid groups to adsorb water molecules into which the peptides are adsorbed). In one embodiment, the solid phase comprises a strong cation exchanger (SCX). Further examples of solid phases that are useful include ion exchange resins such as a strong cation exchanger that uses sulfonic acid groups to adsorb the peptides, a weak cation exchanger that uses carboxylic functionality to adsorb the peptides, a strong anion exchanger that uses quaternary amines to adsorb the peptides, and/or a weak anion exchanger that uses secondary or tertiary amines to adsorb peptide. Reversed phase resins such as C2, C4, C8, and C18 or the polymeric reversed phase solid phases (e.g., Poros R1, R2, and/or R3, and PLRP-S type phases) are also useful for adsorbing peptides.

In another aspect, the invention features a method for preparing a peptide methyl ester. The method includes the step of: providing a first solution comprising diazomethane and a solvent, wherein the solvent is miscible in water; and mixing the first solution with a second solution, wherein the second solution is aqueous and comprises a peptide species, to thereby form a methyl ester of the peptide species.

The term “miscible in water” refers to solvents that are freely soluble in water (e.g., solvents that have a solubility of 80-100% in water). Examples of solvents that are useful for the method include methanol, ethanol, isopropanol, acetonitrile, ethanolamine and triethanolamine.

In one embodiment, the method further includes sequencing the peptide species after formation of the methyl ester.

In another aspect, the invention features a method of determining the relative quantity of a peptide species in a mixture of peptides. The method includes the steps of: (a) providing a first sample including a first population of the peptide species, wherein the concentration of the peptide species in the first sample is less than 1 nM; (b) esterifying the first population of the peptide species to form a first population of peptide esters; (c) providing a second sample including a second population of the peptide species, wherein the concentration of the peptide species in the second sample is less than 1 nM; (d) esterifying the second population of the peptide species with an isotopically enriched reagent to form a second population of isotopically labeled peptide esters; (e) mixing the first population of peptide esters with the second population of isotopically labeled peptide esters to form a mixture; (f) separating the mixture into a plurality of fractions; (g) analyzing a fraction with a mass spectrometer to obtain a first signal for the first population of peptide esters and a second signal for the second population of isotopically labeled peptide esters; and (h) determining the relative quantity of the peptide species in the first sample as compared to the second sample.

In one embodiment, the first population of peptide esters includes peptide methyl esters. In another embodiment, the second population of isotopically labeled peptide esters includes peptide methyl esters.

The second population of the peptide species can labeled with a stable isotope such as deuterium, carbon-13, nitrogen-15 and oxygen-18. In one example, the second population of the peptide species is esterified using a solution containing an alcohol. In another example, the second population of the peptide species is esterified using a solution containing a substituted alcohol.

In one embodiment, the first sample and second sample contain biological material derived from the same cell type or tissue type. In another embodiment, the first sample and second sample contain biological material derived from different cell types or tissue types.

The determining step of the method can include ascertaining the ratio of hydrogen in the first population of peptide esters to deuterium in the second population of isotopically labeled peptide esters.

In addition, the method can further include sequencing the peptide species after determining the relative quantity of the peptide species.

Methods of obtaining a preparation of peptides are well known and include: culturing transformed host cells under culture conditions suitable to express the encoded peptides and purifying the resulting peptides using known purification process, such as gel filtration and ion exchange chromatography. The purification of peptides may also include using an affinity column containing agents that bind to the peptides; one or more column steps over such affinity resins as concanavalin A-agarose, heparin-toyopearl® or Cibacrom blue 3GA Sepharos®; one or more steps involving hydrophobic interaction chromatography using such resins as phenyl ether, butyl ether, or propyl ether; and immunoaffinity chromatography.

Additionally, one or more reversed-phase high performance liquid chromatography (RP-HPLC) steps employing hydrophobic RP-HPLC media, e.g., silica gel having pendant methyl or other aliphatic groups, can be employed to further purify the peptides. Some or all of the foregoing purification steps, in various combinations, can also be employed to provide a substantially homogenous isolated recombinant peptide.

Alternately, a preparation of peptides may also be produced by known conventional chemical synthesis. Methods for constructing peptides by synthetic means are known to those skilled in the art and include solid phase synthesis schemes that employ strategies that build peptides one amino acid residue at a time from the C-terminal end with side chains protected with either 9-fluorenylmethyloxycarbonyl (FMOC) groups, or t-butyloxycarbonyl (t-BOC) groups.

Peptides may be isolated from any sample of interest. Samples may be obtained from vertebrate or invertebrate cells, tissues, or organs, or alternately from cells grown in culture under either control or experimental conditions. The choice of a sample of interest will thus depend on the experimental protocol of the investigator and objective of the investigation. Therefore, in this aspect of the invention, a comparison of results obtained from a first sample to a second sample can involve a first sample that is from the same cell, tissue or organ type as the second sample. Alternately, the comparison can involve a first sample from a different cell, tissue or organ type as the second sample. Comparison of a first sample from the same cell, tissue or organ as the second sample might be done to determine the effects of an experimental condition as compared to a control sample, for example, while comparison of a first sample from a different cell, tissue or organ as the second sample might be carried out to determine how different cells, tissues or organs respond to an experimental condition.

In one aspect of the invention, peptides can be methylated using the methods described herein. Methylation of peptides can be carried out using, for example, alcohols or substituted alcohols, as described herein.

“A reagent comprising molecules containing methyl groups” refers to chemicals having reactive (—CH₃ or ═CH₂) groups. Examples of such reagents include diazomethane, methanol, and methanolic HCl.

“An isotopically enriched reagent” refers to any isotope of a chemical element that is not subject to rapid degradation, e.g., an isotope that has a long half-life. Examples of such useful isotopes include deuterium, carbon-13, nitrogen-15 and oxygen-18.

A “mixture” of peptide esters and isotopically labeled peptide esters refers to a solution that contains specific amounts of the first component (e.g., peptide methyl esters) and the second component (e.g., isotopically labeled peptide methyl esters). Varying the components of the mixture will depend on the experimental conditions being tested and under the control of the skilled artisan. Examples of mixtures that are useful in the invention include 4:1, 3:1, 2:1, 1:1, 0.5:1, 0.3:1, 0.25:1.

“Separating the mixture into fractions” refers to any method of fractionating the solution. Methods of fractionation of peptide solutions are well known in the art and include reversed phase, ion exchange, HILIC, and normal phase chromatography.

In one aspect, the invention features the use of peptide esterification in conjunction with deuteroesterification. These methods can be used, for example, to detect peptides, e.g., novel peptides in antigen pulsed cell lines, to quantitate expressed protein tag (EPT) levels for application to proteomic studies, and/or to permit peptide sequence elucidation.

In another aspect, the invention features a method of fractionating peptides. The method includes the steps of: (a) providing a sample comprising a plurality of different peptides; (b) adsorbing the peptides onto a strong cation exchanger; (c) selectively desorbing a first subset of the peptides by the action of a first mobile phase; (d) adsorbing the first subset of peptides onto a reversed phase HPLC column; (e) selectively eluting a second subset the peptides from the reversed phase HPLC column by a second mobile phase that develops an increasing acetonitrile concentration gradient; and (f) collecting peptide fractions eluted from the reversed phase HPLC column.

In one example of this method, the reversed phase HPLC column is a micro-HPLC column containing both a reversed phase and a strong cation exchanger stationary phase, and the second mobile phase contains both an acetonitrile gradient and a pH gradient.

The method can optionally further include sequencing a peptide contained in a peptide fraction by mass spectrometry.

In another aspect, the invention features a peptide separation system including: a column containing an ion exchange stationary phase and a reversed phase HPLC phase, wherein the ion exchange stationary phase has strong cation exchange characteristics, and wherein the reversed phase HPLC has C2, C4, C8, C18 or polymeric characteristics; and a mobile phase gradient containing an acetonitrile gradient and a pH gradient. The peptide separation system can optionally include a mass spectrometer, e.g., an on-line mass spectrometer wherein that receives peptide eluted via the mobile phase gradient.

In another aspect, the invention includes a method, e.g., a method described herein, of sequencing peptides in a sample at sub-femtomole levels of sensitivity. One method includes the steps of: (a) isolating a first population of peptides from a first sample; (b) esterifying the first population of peptides with a reagent containing molecules containing alkyl groups to form peptide esters; (c) isolating a second population of peptides from a second sample, wherein the second sample is substantially identical to the first sample; (d) mixing the peptide esters of (b) with the second population of peptides from (c) to form a mixture; (e) separating the mixture into fractions; (f) analyzing the fractions with a mass spectrometer to obtain a first peptide map for the peptide esters and a second peptide map for the second population of peptides; and (g) sequencing a population of peptides in the fractions by comparing the first peptide map obtained for the peptide esters with the second peptide map obtained for the second population of peptides.

“Sub-femtomole levels of sensitivity” refers to the analysis of microliter aliquots of peptide mixtures that contain individual components that are at concentrations below 10⁻⁹ M (1 nM). It is an advantage of the invention to be able to sequence peptides at such low levels of detection.

Alkyl group refers to a hydrocarbon, comprised of carbon and hydrogen atoms that may be of various carbon chain lengths and contain various branches of various carbon chain lengths. Carbon chain lengths of 1 to 4 and branch chain lengths of 0-4 carbon atoms can be used in the methods described herein.

“Substantially identical” is meant to refer to a second sample that has been derived from the same type of cell, tissue or organ of interest as a first sample at the same time and under the same conditions.

In this aspect of the invention, peptides can be esterifieded using the methods described herein.

A “peptide map” refers to the pattern of signals that is generated by HPLC separation of peptides

In another aspect, the invention includes a method of sequencing peptides in a sample at femtomole levels of sensitivity. The method includes the steps of: (a) isolating a first population of peptides from a first sample; (b) esterifying the first population of peptides with a reagent comprising molecules containing alkyl groups to form peptide esters; (c) isolating a second population of peptides from a second sample, wherein the second sample is substantially identical to the first sample; (d) esterifying the second population of peptides with an isotopically enriched reagent comprising molecules containing stable isotopes to form esters that are labeled with a stable isotope; (e) mixing the peptide esters of (b) with the esters that are labeled with a stable isotope of (d) to form a mixture; (f) separating the mixture into fractions; (g) analyzing the fractions with a mass spectrometer to obtain a first peptide map for the peptide esters and a second peptide map for the esters that are labeled with a stable isotope; and (h) sequencing a population of peptides in the fractions by comparing the peptide map obtained for the peptide esters with the second peptide map obtained for the esters that are labeled with a stable isotope.

In this aspect of the invention, esterification of peptides can be accomplished with, for example, methods using alcohols or substituted alcohols, as described herein. Forming esters that are labeled with stable isotopes can be performed by the methods described herein.

Also within the invention is a system for fractionating peptides using combined multi-modal HPLC separation mechanisms including: (a) a combination of ion exchange chromatography with reversed phase chromatography; (b) an ion exchange stationary phase, wherein the ion exchange stationary phase has strong cation exchange characteristics; (c) a reversed phase HPLC stationary phase, wherein the reversed phase HPLC stationary phase has C2, C4, C8, C18 or polymeric characteristics; (d) a mobile phase comprising acetonitrile, wherein the mobile phase promotes a mechanism by which peptides are fractionated by their hydrophobic character; and (e) peptide-ester fractions which are collected for analysis by a LC/MS/MS.

“Reversed phase HPLC stationary phase that has C2, C4, C8, C18 or polymeric characteristics” refers to a solid particle that imparts hydrophobic character to adsorb peptides or a solid particle that has covalently linked to it functional groups that impart hydrophobic character to adsorb peptides. “Cation exchange properties” refers to a solid particle that has anionic character that attracts the positively charged molecules.

For the sake of this application, the terms “a strong cation exchanger” and “a strong cation exchanger stationary phase” are used interchangeably. The terms refer to a column of particles that adsorb peptides by a cation exchange mechanism.

“Mobile phase” refers to a solvent system that promotes analyte desorption from a chromatographic stationary phase. Examples of mobile phases useful in the invention include compositions of acetonitrile and water for reversed phase chromatography and HILIC separation and aqueous solutions of low to high pH for SCX chromatography.

In another aspect, the invention includes a method of fractionating peptides, including the steps of: (a) providing a sample of peptides; (b) adsorbing the peptides onto a strong cation exchanger in the presence of a high acetonitrile concentration; (c) selectively desorbing the peptides by the action of mobile phases that develop a gradient of increasing water concentration; (d) fractionating the peptides by the strong cation exchanger; (e) re-adsorbing the peptides onto a reversed phase HPLC column; (f) selectively eluting the peptides from the reversed phase HPLC column by mobile phases that develop an increasing acetonitrile concentration gradient; and (g) collecting peptide fractions for analysis by a LC/MS/MS.

A “high acetonitrile concentration” is meant to denote concentrations of acetonitrile that are above 70%. Examples of high acetonitrile concentrations include 75, 80, 85, 90, 95 and 100%.

In another aspect, the invention includes a method of sequencing peptides, including the steps of: (a) fractionating a sample of peptides by a method described herein; (b) adsorbing the peptides onto a micro-HPLC column comprising a reversed phase and a strong cation exchanger stationary phase; (c) selectively eluting the peptides from a mixed phase column by the action of mobile phases, wherein the mobile phases develop an increasing pH and an increasing acetonitrile concentration; (d) analyzing the peptides with a mass spectrometer; and (e) sequencing the peptides that are detected by the mass spectrometer by tandem mass spectrometry.

In another aspect, the invention includes a method of sequencing peptides, including the steps of: (a) fractionating a sample of peptides by a method described herein; (b) adsorbing the peptides onto a micro-HPLC column comprising a strong cation exchanger stationary phase; (c) selectively eluting the peptides from a strong cation exchange phase by the action of mobile phases, wherein the mobile phases develop an increasing pH; (d) re-adsorbing the peptides that were eluted from the strong cation exchange phase onto a reversed phase; (e) separating the peptides using mobile phases that deliver an increasing acetonitrile concentration; (f) analyzing the peptides with a mass spectrometer; and (g) sequencing the peptides that are detected by the mass spectrometer by tandem mass spectrometry.

As used herein, the term “a mixed phase column” refers to a column that is prepared with two or more stationary phases of different chemical properties. The terms “mixed phase column” and “mixed bed column” are used interchangeably. “Tandem spectrometry” refers to multiple stages of mass spectrometry (e.g., mass spectrometry-mass spectrometry (MS/MS) refers to isolation of a precursor ion and fragmentation to products).

“A precursor ion” is a signal that is generated by a mass spectrometer, which commonly defines the molecular weight of the analyzed substance.

“Fragmentation products” that result from MS/MS studies are signature signals that define the chemical composition of an analyzed substance.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 depicts a comparison of MALDI-TOF-MS data acquired for a native mixture of HLA-A1 presented peptides. Peptide maps for native and methylated peptides are compared.

FIG. 2 depicts the quantification of relative peptide concentration (RT=Peptide Retention Time; AA=Peak Area Measurement as made by a Quantification Algorithm Supplied by the Instrument Vendor)

FIG. 3 demonstrates the effects of isotope labeling of peptide through esterification. This information can be used to determine the amino acid sequence of a peptide. Any fragment ion that increases in m/z value by 3 daltons contains an acidic residue. Those fragment ions that contain more than one acidic residue increase in m/z by 3 daltons multiplied by the number of acidic residues (there are no such fragment ions displayed in FIG. 3). The figure depicts a comparison of methylated and deuteromethylated LC/MS/MS Spectra for NTDHQTQLLY (SEQ ID NO: 1) as collected under the peaks shown in FIG. 2.

FIG. 4 shows a schematic of the LC/MS/MS system in which a SCX trapping a phase is decoupled from a reversed phase micro-capillary column.

FIG. 5 shows a typical flow of work for identifying and quantifying peptides from two complex mixtures of peptides following esterification with standard reagents (one peptide mixture, e.g., from a control cell line), and reagents that are labeled with a stable isotope (a second peptide mixture, e.g., from a stimulated cell line).

### DETAILED DESCRIPTION

Peptide Esterification Methods

The invention includes methods for the esterification of a peptide species or a population of peptides. According to these methods, peptides that contain an acidic residue or free acid C-terminus can be derivatized.

In one method, an acidified alcohol solution is used for the esterification of a peptide species. The method includes, for example, the use of an acidified alcohol such as methanolic HCl to generate a peptide ester, e.g., a peptide methyl ester. Additionally, other alcohols (such as ethanol, propanol, or isopropanol) or substituted alcohols (such as aminoethanol) can be used to generate a reagent for esterification of acidic residues. Following the mixing of the acidified alcohol solution with a sample containing the peptide species, the concentration of the peptide species is preferably less than 1 nM. In some examples, the concentration of the peptide species is less than 100 pM, 10 pM, 1 pM, 100 fM, 10 fM, or 1 fM.

In a second method, diazomethane is collected in a solvent, preferably a solvent other than diethyl ether. The solvent is preferably miscible in water. The solvent is mixed with a second solution that contains a peptide species, to thereby form a peptide methyl ester of the peptide species. The concentration of the peptide methyl ester of the peptide species is preferably less than 1 nM. In some examples, the concentration of the peptide methyl ester of the peptide species is less than 100 pM, 10 pM, 1 pM, 100 fM, 10 fM, or 1 fM.

According to methods described herein, an acidic residue (e.g., aspartic acid or glutamic acid) and/or a carboxy terminal acid group is modified by combination with, e.g., (CH₂)₂NH₂. Hence, an acidic functional group is replaced to effectively change the chemical properties of a peptide. Furthermore, converting a singly charged peptide into a doubly charged peptide can aid in peptide sequence elucidation. For example, the MS/MS spectrum of a peptide that is derivatized with aminoethanol is expected to contain both b (N-terminal) and y (C-terminal) sequence ions. Other reagents that have specific functionality to aid sequence elucidation and/or peptide isolation can also be used in the methods of this invention. These include reagents that have a quaternary amine in addition to the alcohol group that is required for ester formation with the acidic residues of the peptide. Examples include trialkylammonium-substituted alcohols such as trimethyl-ammonium ethanol. Additionally, alcohols that have specific functionality, such as biotin or histidine that can be used to isolate and purify peptides can be used.

An esterification reaction can be performed on a solid phase using, for example, an acidified alcohol method in conjunction with solid phase such as a hydrophilic chromatography (HILIC) phase (e.g., a strong cation exchanger). By this approach, the peptides can be loaded onto the stationary phase in a suitable solvent (e.g., acidified water with or without a solvent such as a polar organic solvent, e.g., acetonitrile or methanol). The acidified alcohol is then passed through the column where the esterification reaction occurs. Subsequently, the esterification reagent is washed from the column and the peptides are eluted in a suitable way using either HILIC mobile phases (such as an acetonitrile to water gradient), salt or pH gradient. The derivatized peptides can be further fractionated and then analyzed, or analyzed directly.

Chromatography

Esterification of a peptide sample together with mixing a second peptide sample increases the complexity of peptide mixtures, e.g., HLA-presented peptide mixtures. In addition, peptide signals that were of low abundance in the native peptide spectrum may be detected with increased intensity. This increases the probability of detecting isobaric peptides and overlapping peptide isotope signals. In this regard, enhanced separation may be required. Multi-modal separations using combinations of separation techniques can be used. Such techniques can include the following chromatographic and electrophoretic modes of separation to increase peptide resolution during fractionation: cation-exchange; anion-exchange; HILIC (hydrophilic chromatography); normal and reverse phase; metal ion affinity; hydrophobic interaction chromatography; capillary electrophoresis; and capillary electrochromatography. Additionally, further dimensions of separation on-line with a mass spectrometer can be performed. Combinations and mixed bed columns are used in the methods described herein to enhance peptide separations and maximize the identification of peptides by LC/MS/MS. One such approach uses a mixed bed column containing a strong cation exchange (SCX) and reversed phase with a simple combined acetonitrile/pH gradient to enhance peptide resolution and therefore aid collection of sequence specific data for individual peptides. Another approach uses the same combined acetonitrile/pH gradient system with separate columns of strong cation exchange material and reversed phase stationary phase, to enhance peptide resolution and aid peptide sequence identification.

In some aspects, the invention features systems and methods for fractionating, separating, and sequencing peptides. Peptide and protein samples are isolated from cell lines, tissue samples, or intact organisms. These samples are manipulated in ways known to skilled artisans to generate peptide mixtures. For example, cell receptors may be isolated and treated in such a way as to release the peptides to which they are complexed. Alternatively, a protein mixture may be digested either enzymatically or chemically to produce a mixture of peptides. Similar peptide mixtures can be generated from stimulated cells, abnormal tissues or treated organisms.

However the peptide mixtures may be generated, one of the peptide mixtures can be esterified with standard (non-isotpoic) reagents and the other peptide mixture can be esterified with reagents that contain a stable isotope. Subsequently, the esterified and isotope-esterified peptide mixtures are mixed. This combined mixture is substantially more complex than each extract alone, and single mode chromatographic separation is typically inadequate to sufficiently fractionate the peptides to allow maximal component identification. In this aspect of this invention, multi-modal separation strategies for fractionating peptides have been implemented. Coupling of one or more chromatographic modes together significantly simplifies peptide mixtures, thereby increasing the ability to identify more components of a complex mixture. Many modes of chromatography are known to separate peptides. These include normal and reversed phase in which hydrophobic character is exploited to fractionate individual components of the mixture, hydrophilic interaction chromatography (HILIC) in which the hydrophilic character of peptides is exploited to resolve components, and ion exchange chromatography where the ionic character of peptides in different solutions can be used to separate and fractionate these biopolymers. Other modes including affinity chromatography, such as immobilized metal chelation chromatography (IMAC), immunoaffinity chromatography, and size exclusion chromatography are also useful to separate complex mixtures of peptides. However, many peptides of a complex mixture share similar chemical properties, hence, a single mode of chromatography has a limited capacity to completely resolve a mixture of these biopolymers. More appropriate is to use two or more modes of separation in series. For example, coupling ion exchange chromatography with reversed phase chromatography uses both the ionic and hydrophobic characters of the separation techniques to effect the isolation of target peptides.

The ion exchange column “bins” the peptides according to charge, while the reversed phase separation is used to further resolve and simplify collected fractions. Peptides can be “binned” by ion exchange chromatography using the effects of an increasing salt concentration gradient. For cation exchange chromatography, peptides can be displaced from the solid phase By the action of a small cation (such as sodium, potassium, ammonium, etc.) that has a higher affinity for the solid phase than the peptide. The number of charges that reside on a peptide dictates the strength of its interaction with ion exchange supports, with a higher number of charges proportionately increasing the concentration of salt required to elute the peptide from the column. In this way, peptides that are singly charged in solution are separated from those of double charge, which are in turn separated from those of higher charge.

Alternatively, peptides can be eluted from a cation exchange stationary phase by the action of a pH gradient. Peptides have the ability to take on both positive and negative charge, depending upon the solution in which they are dissolved. In acidic solutions, peptides will be positively charged to varying degrees depending upon the chemical nature of the amino acid residues from which they are comprised. These cationic species adsorb to cation exchange resin. As the pH of the solution that is passed through the cation exchange column is increased, the positive charge that resides on the peptides changes so that ultimately the peptides become negatively charged, at which time they are eluted from the column. Alternatively, peptides can be separated according to their hydrophobic character by reversed phase chromatography, in which a water to organic solvent (e.g., acetonitrile) gradient is use to selectively separate and elute peptides from the column. Hence, coupling the two modes of chromatography (cation exchange with reversed phase) enhances peptide separation. The peptides “binned” by charge by the cation exchange chromatography are subsequently further resolved using reversed phase chromatography, and eluted from the second column in the order of increasing hydrophobicity. Likewise, other modes of chromatography are complementary. For example, hydrophilic chromatography generally separates peptides in accordance with their different hydrophilic character. Hence, coupling HILIC and reversed phase chromatography enhances the separation of complex peptide mixtures. Ultimately, as mentioned above, a further dimension of chromatography on-line with mass spectrometric analysis of the peptide fractions is preferred. Here, an orthogonal chromatographic approach is desirable. A reversed phase chromatography that uses an acetonitrile gradient modified to a basic pH (rather than acidic pH) has been found to be a useful orthogonal mode of chromatography to enhance the analysis of peptide fractions that were generated as described above.

The application of such alkaline modified reversed phase conditions enhances the resolution of peptide mixtures, and an elution time window of approximately 14 minutes (during which the peptides were eluted from the column) for reversed phase separated peptide fractions. Under acidic modified reversed phase conditions this elution time window was at best 2 minutes, with all peptides of the sample eluting over this short duration. Furthermore, a mixed bed column that simultaneously encompasses multiple modes of chromatography with a binary solvent gradient to promote increasing character of two chemical properties is desirable for this purpose. In one example, a combined SCX, reversed phase column with a gradient that increases pH and organic character of the mobile phase has proved useful for enhancing the separation of peptides on-line with the mass spectrometer and has improved peptide sequencing efforts. In a second example, decoupling the SCX phase from the reversed phase column has further enhanced peptide-sequencing efforts by micro-capillary LC/MS/MS.

Applicability of Esterification to Proteomics

The methods described herein are applicable to the study of peptides and proteins, e.g., peptides and proteins extracted from in vivo and in vitro sources (e.g., blood, cerebrospinal fluid (CSF), urine, feces, tissues, or cultured cells). Protein biochemistry studies of human and non-human organisms, including plants, fungi, mammals, birds, fish, amphibians, eukaryotic parasites, bacteria, viruses, and other organisms, are all possible uses of the invention. Advantages include the ability to measure and identify a wide variety of peptides and proteins, including peptides and proteins that do not contain an amino acid residue that can be alkylated (e.g., do not contain a cysteine residue).

Other important uses of the invention include mapping the surface and other solvent accessible sites of a protein. For example, reaction of the protein with the methylating reagent prior to digestion can yield information regarding three-dimensional structure of a protein. A specific example is probing the active site of a protein (e.g., a serine protease which has an aspartic acid residue at the active site). This approach could also be used to inhibit an enzyme such as a serine protease. In these methods, complex peptide mixtures can be generated from isolated proteins using specific proteases such as trypsin, chymotrypsin, lysine endopeptidase, endoproteinase Lys-C, endoproteinase Asp-N, endoproteinase Glu-C, or by chemical methods. Protein mixtures can be pre-fractionated or taken from total cell lysates or generated by any other known approaches (e.g., chromatography or gel electrophoresis). Additionally, the esterification methods can be performed either before or after the enzymatic or chemical digestion. Immobilized enzyme columns can also be used to generate peptide fragments from protein samples.

The following are examples of the practice of the invention. They are not to be construed as limiting the scope of the invention in any way.

### EXAMPLES

### Example 1

### Methylation of Peptides Using Diazomethane

Acetonitrile (0.5-3 mL) was added to the outer tube of an Aldrich diazomethane generator. Diazald® (Aldrich (Steinheim, Germany)) (N-methyl-N-nitroso-p-toluenesulfonamide; 0.2-0.5 g) was dissolved in a solution containing diethyl ether (1 mL) and carbitol (2-(2-ethoxyethoxy) ethanol; 1 mL) and placed in the center tube of the generator. The generator was assembled and placed in an ice bath. Sodium hydroxide (30% w/v in water; 1.5 mL) was dispensed drop-wise through the rubber septum of the apparatus using a 22-gauge needle to prevent loss of diazomethane around the shank. The rate of addition was less than 1 drop/5 seconds to prevent pressure build-up in the apparatus. The generated diazomethane dissolved in the acetonitrile that was contained in the outer tube of the apparatus (this took on a bright yellow color as the concentration of diazomethane was increased). The apparatus was gently shaken by hand every 10 min to ensure complete reaction. The maximum yield of diazomethane was obtained in ˜35 -60 min. The generated acetonitrile solution of diazomethane was kept on ice until it was used.

The diazomethane-rich acetonitrile solution was added to aqueous peptide solutions. Further reagent was added until a yellow color persisted in the sample. At this time the excess reagent was removed and the sample was concentrated to the original volume in a Savant SpeedVac concentrator.

### Example 2

### Methylation of Peptides Using Deuterodiazomethane

Deuterodiazomethane was generated in the same manner as diazomethane, with substitution of reagents. Diazald-N-methyl-d3 (0.3-0.4 g) was dissolved in anhydrous diethyl ether (1 mL) and carbitol-d (1 mL) and placed in the center tube of the Aldrich diazomethane generator. Acetonitrile (0.5-3 mL) was placed in the outer tube of this apparatus, and the apparatus was assembled and immersed in an ice bath. Sodium deuteroxide (30 wt %, 1.5 mL) was added drop-wise, and the reagents were reacted as described above.

The deuterodiazomethane-rich acetonitrile solution was added to aqueous peptide solutions. Further reagent was added until a yellow color persisted in the sample. At this time the excess reagent was removed and the sample was concentrated to the original volume in a Savant SpeedVac concentrator.

### Example 3

### Esterification of Peptides by Methanolic HCl

Methanolic hydrochloric acid was prepared by dissolving acetyl chloride in dry methanol. Concentration and volume of reagent, time and temperature of reaction were optimized to increase the yield of peptide methyl esters, and ensure quantitative esterification of all acidic functional groups. Optimal conditions for peptide esterification were found to be the addition to a completely dry peptide sample of 30-80 μL of reagent prepared by dissolving acetyl chloride (120-180 μL) in methanol that was dried over anhydrous sodium sulfate (0.8-1.2 mL). A reaction time of 45 minutes at a temperature of 37° C. yielded quantitative methylation of acidic residues of a naturally derived HLA-presented peptide mixture. Following reaction, the methylated peptide mixture was again reduced to dryness prior to reconstitution in an aqueous solvent (water/acetonitrile/trifluoroacetic acid 95:5:0.04 v/v/v).

### Example 4

### Esterification of Peptides by Deuterated Methanolic HCl

Deuterated methanolic hydrochloric acid was prepared by dissolving deuterated acetyl chloride (120-180 μL) in d₄-methanol (0.8-1.2 mL) that was dried over anhydrous sodium sulfate. Peptide samples were taken to complete dryness in a Savant SpeedVac™ and reacted for 45 minutes at 37° C. with 30-80 μl of deuterated reagent. Following reaction, the peptide mixture was again reduced to dryness prior to reconstitution in an aqueous solvent (e.g., water/acetonitrile/trifluoroacetic acid 95:5:0.04 v/v/v).

### Example 5

### Mass Spectrometry—Matrix Assisted Laser Desorption/Ionization Time of Flight Mass Spectrometry (MALDI-TOF-MS)

MALDI-TOF-MS analysis of esterified peptide mixtures was performed by co-crystallizing an aliquot of a sample with a light-absorbing matrix (such as 2,5-dihydrobenzoic acid, DHB; or 4-hydroxy cinnamic acid, α-cyano). Specifically, an aliquot (0.5-1.0 μL) of a matrix solution comprising DHB (5-15 mg/mL) in an aqueous solution (e.g. water/acetonitrile/trifluoroacetic acid 70:30:0.1 v/v/v) was placed onto the instrument (Voyager Elite XL from PerSeptive Biosystems, Framingham, Mass.) sample stage and allowed to dry. Once the matrix solution had dried, an aliquot (0.5-1.0 μL) of sample was applied to the top of the dry matrix spot. When α-cyano was used, the procedure for sample preparation was similar except the sample was first applied to the instrument sample stage and dried. Then a solution of α-cyano at a concentration of (5-15 mg/mL) dissolved in a solution such as water/acetonitrile/trifluoroacetic acid 70:30:0.1 v/v/v was added to the sample spot and dried. Spectra were collected by a means known to those skilled in the art (e.g., ions were generated by laser light's hitting the sample, the ions were then accelerated and allowed to drift down a flight tube that facilitates their separation. Signals that are detected at the detector are collected on a computer where ion flight times are converted to mass to charge (m/z) values by comparison of sample data with calibrated time to m/z conversion values generated by analysis of known entities).

### Example 6

### Mass Spectrometry—Liquid Chromatography/Tandem Mass Spectrometry (LC/MS/MS)

LC/MS/MS is achieved by coupling a separation technique, e.g., reversed phase HPLC to an ion trap mass analyzer. However, other mass analyzers such as but not limited to a triple quadrupole instrument, magnetic sector, fourier transform ion cyclotron resonance, quadrupole time-of-flight, or a hybrid of these analyzers could be coupled to on-line HPLC separations though an electrospray interface, and used for this application. Aliquots (0.5-10 μL) of samples were diluted in suitable aqueous solutions (e.g. 5-15 μL of an aqueous solution containing acetonitrile and trifluoroacetic acid (TFA). A suitable composition of solvent for this application is water/acetonitrile/trifluoroacetic acid 95:5:0.04 v/v/v. The whole of the resulting mixture is injected on to a miniaturized peptide trap that contains a small bed volume (0.5 μL) of HPLC stationary phase (such as polymeric support, C2, C4, C8, C18, metal affinity support, ion exchange media or the like) that is used in place of a sample loop in a HPLC injector valve. Peptides elute from this phase by action of the gradient that flows across it and are further adsorbed and separated on a micro-capillary HPLC column (e.g., 20-100 μm id. column that is packed with a stationary phase that is suitable for separating peptides such as polymeric support, C2, C4, C8, C18, metal affinity support, ion exchange media). Combinations of support material such as ion exchange material (e.g., SCX material) followed by reversed phased support (e.g. C2, C4, C8, or C18 material) can also be used for this purpose. Peptides are separated and eluted from the column due to the effects of a solvent gradient (such as increasing the concentration of acetonitrile after starting with a predominantly aqueous mobile phase). Such mobile phases can be modified with either acids or bases to change peptide separation characteristics. A typical mobile phase gradient can be 5-100% acetonitrile in 15 minutes with both mobile phases modified with formic acid (0.1-0.5% by volume), acetic acid (0.5-2.0% by volume) or TFA (0.01-0.05% by volume). A mixed acid modified mobile phase system (e.g., 0.5% acetic acid and 0.04% TFA) can also be used to modify peptide separation characteristics. Alternatively, mobile phases can be modified with bases such as ammonium hydroxide (1-10 mM) to effect different peptide separations. The mass spectrometer can be used in targeted mode, where specific peptide signals are isolated and fragmented, and data dependent mode of operation. In the latter mode, knowledge of sample composition is not required as specific peptide signals are isolated and fragmented when they are more intense than an operator defined threshold.

### Example 7

### Quantitative Methylation of a Naturally Derived HLA Presented Peptide Mixture

Quantitative methylation entails the reaction of substantially all of the acidic functional groups in all of the peptides of a protein mixture. Hence a peptide in a native mixture is converted by reaction to a fully methylated product at the end of the reaction. While methylation is an example described, quantitative deuteromethylation can also be used, since the chemical nature of reactions (e.g., rates and mechanisms) and reaction conditions are the same. Deuteromethylation can use isotopically labeled reagents that include deuterium, carbon 13, nitrogen 15 and/or oxygen 18 isotopes. Quantitative methylation of a peptide mixture was demonstrated by the following method.

An aliquot (10 μL) of a reversed phase HPLC fraction of HLA-A1 presented peptides derived from an immortalized human B-cell line was reduced to dryness and reacted with methanolic HCl as described above. MALDI-TOF-MS was used to analyze the native peptide mixture and the sample generated after treatment with the optimized methanolic HCl methylation procedure. Following reaction, the peptide mixture was again reduced to dryness prior to reconstitution in an aqueous solvent (water/acetonitrile/trifluoroacetic acid 95:5:0.04 v/v/v). Samples were prepared for MALDI-TOF-MS analysis by co-crystallization of each peptide mixture with a solution of 2,5-dihydroxybenzoic acid (DHB) in an aqueous solution (water/acetonitrile/trifluoroacetic acid 70:30:0.1 v/v/v). The results of these analyses (FIG. 1) indicated that the procedure yielded quantitative methylation of the acidic groups. All of the peptide signals, except one (m/z 1029.5), detected in the mass spectrum of the native peptides were converted in their entirety to their esterified analogs. This was determined by detection of peptide responses of increased m/z in the spectrum collected after methylation of the sample. There was also no signal due to the native peptides in this mass spectrum. As shown in Table 1, these data enabled elucidation of the number of acidic residues (the C-terminus as well as asparagine and glutamine residues) in each peptide. Furthermore, there was no appreciable loss of peptide (as determined by comparison of peak responses in the two MALDI-TOF-MS spectra shown in FIG. 1). One peptide was observed only after methylation (see Table 1). It was concluded that this peptide signal corresponded to deamidation (conversion of glutamine to glutamic acid or asparagines to aspartic acid) in the peptide of MH⁺=1232.6 followed by three sites of methylation.

TABLE 1
Correlation of Detected Peptide Signals Between the Native and
Methylated MALD-TOF-MS Spectra Shown in FIG. 1 Enabled
Determination of the Number of Acid Residues
in HLA-A1 Presented Peptides

| m/z Native | m/z Methylated | Number of Sites of |
|---|---|---|
| Peptide | Peptide | Methylation |
| 1029.5 | 1029.7 | 0 |
| 1070.5 | 1098.7 | 2 |
| 1137.6 | 1165.7 | 2 |
| 1169.8 | 1211.8 | 3 |
| 1191.5 | 1219.8 | 2 |
| 1225.7 | 1239.7 | 1 |
| 1232.6 | 1260.8 | 2 |
| 1254.6 | 1278.8 | 2 |
| 1286.3 | 1314.7 | 2 |
| 1296.6 | — | Contaminant |
| — | 1275.7 | Deamidation of Q to E or |
| | | N to D plus 3 sites of |
| | | methylation |

### Example 8

### Peptide Quantification by Isotope Dilution Following Methylation and Deuteromethylation

The methods described herein can be used to determine the relative quantity of a peptide in a cell line or sample, e.g., a tissue sample, as compared to the amount of the same peptide in a different cell line or sample. By this approach the methyl ester of a peptide extract from one cell line or tissue sample and the deuteromethyl ester of the other are prepared and mixed prior to peptide isolation, fractionation and analysis. The relative amounts of the methyl and deuteromethyl analogs determine the relative amounts of the native peptide in the cell line or tissue extracts. This has been exemplified as follows.

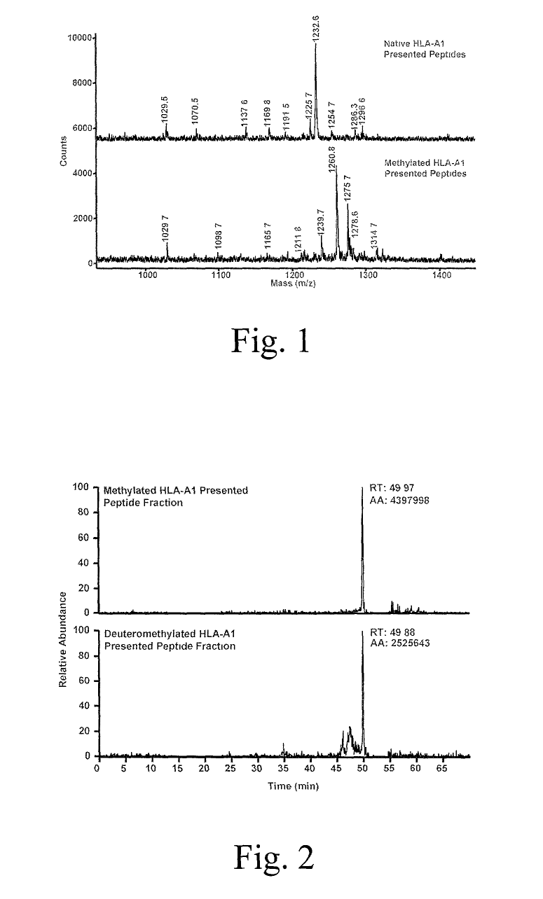

An aliquot (10 μL) of a reversed phase HPLC fraction of HLA-A1 presented peptides derived from an immortalized human B-cell line was reduced to dryness and reacted methanolic HCl as described above. A second aliquot (10 μL) of the same HPLC fraction of HLA-A1 peptides was reduced to dryness and reacted with deuterated methanolic HCl. Following reaction, the peptide mixture was again reduced to dryness prior to reconstitution in an aqueous solvent (water/acetonitrile/trifluoroacetic acid 95:5:0.04 v/v/v). The samples were mixed at a rate of two parts of the methylated sample to one part of the deuteromethylated sample and analyzed by targeted LC/MS/MS, isolating the doubly charged precursor ion for doubly methylated peptide (NTDHQTQLLY; SEQ ID NO: 1) that was derived from Rad51, and the doubly charged precursor ion for doubly deuteromethylated analog of the same peptide. Area measurements from under peaks observed in the ion chromatograms reconstructed from the six most abundant fragment ions of both peptides (m/z 468, 477, 524, 533, 952, and 1065 for the methylated peptide and m/z 469, 478, 526, 535, 955 and 1068 for the deuteromethylated peptide) were in good agreement with the ratio of the mixed products (FIG. 2). Specifically, the peak area of the doubly methylated peptide was 4397998, and the peak area measured for the doubly deuteromethylated peptide was 2525643. The ratio of these measurements was 1.74:1, which is in good agreement with the prepared sample (which was prepared at a 2:1, hydrogen: deterium, H:D, ratio).

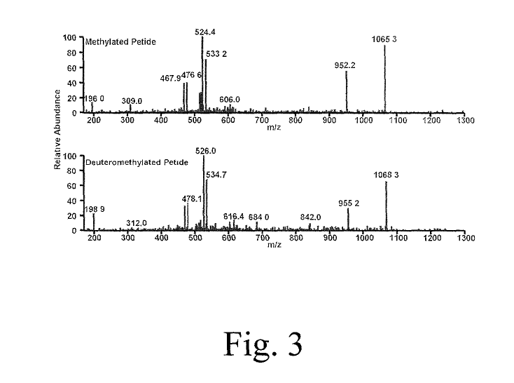

Specific peptide sequence information was also derived from the LC/MS/MS data. Those fragment ions that contained an acidic residue were readily identified by these data as peaks whose m/z value increased by three daltons in the deuteromethylated peptide spectrum (1.5 daltons for doubly charged fragment ions; FIG. 3).

### Example 9

### Esterification for Profile Comparison Maps

An analysis can be directed to the identification of a naturally processed and presented HLA epitope that is derived from an antigen that is specifically expressed by a cell line by any means known to those skilled in the art (including infection, transfection, and incubation).

Naturally expressed peptide pools are isolated by any means known to those skilled in the art (methods for isolating pools of naturally expressed peptides have been described, for example, in U.S. Pat. No. 09/372,380, herein incorporated by reference). For example, the peptide extract of a control preparation can be deuteromethylated and the peptide extract an antigen pulsed cell line can be methylated. These samples are mixed in their entirety prior to peptide fractionation. Fractionated peptides are analyzed by MALDI-TOF-MS. Any peptide signal that does not have an isotopically labeled partner is a target for sequencing by LC/MS/MS. While this specific example describes the use of methylation for HLA peptide research, this invention could be used for comparison of any protein maps.

### Example 10

### Esterification to Enable Peptide Quantification in Studies of Organism Proteomes

An analysis can be directed to the investigation of the effects of stimulants or perturbants on a cell population or a patient subject.

Naturally expressed peptide pools are isolated by any means known to those skilled in the art (methods for isolating pools of naturally expressed peptides have been described for example in U.S. Pat. No. 09/372,380, herein incorporated by reference). The peptide pool extracted from a control cell line is methylated, and the peptide pool extracted from a perturbed cell line is deuteromethylated. Prior to fractionation, the two esterified peptide pools are mixed in their entirety and fractionated using multi-modal chromatographic techniques. The separation techniques used here are novel combinations of HPLC approaches. For example, peptide fractions are generated by strong cation exchange and are further fractionated by reversed phase HPLC. Alternatively, those peptide fractions generated by strong cation exchange can be further fractionated using HILIC, normal phase, anion exchange, metal affinity, hydrophobic interaction chromatography, or by electrophoretic methods (such as capillary electrophoresis and/or capillary electrochromatography).

The fractionated peptides are analyzed by MALDI-TOF-MS. Each peptide is detected with an ion signature that is separated by a multiplicity (that is defined by the number of acidic amino acid residues in a peptide along with its C-terminus) of 2 or 3 daltons (depending upon how the methylation is performed). Those peptides that exhibit a fractional or elevated D/H ratio are targeted for sequencing. By this approach, all peptides are monitored by MALDI-TOF-MS, and knowledge of which peptide to monitor is unnecessary.

In addition to identifying target peptides to sequence, this method can enable quantification of the relative amount of a peptide expressed by the two cell lines. This isotope dilution approach is self-normalizing since both methylated and deuteromethylated peptides are in the same fraction. This can aid proteomic studies by enabling an accurate assessment of the biochemistry (e.g., as reported by naturally processed peptide pools) that has occurred (or is occurring) in the cells/organisms as samples are harvested.

Peptide sequencing efforts can also be aided by this approach since the isotopic label will be detected in all fragment ions except those that do not contain an acid residue and/or the C-terminus. Additionally, for positive ions, the elimination of the negativity of acidic residue (through esterification) may increase the relative response factor of a peptide. This often enhances sensitivity, which also aids sequence elucidation.

### Example 11

### Construction of a Mixed Phase Microcapillary Column

A mixed phase microcapillary column that can be used in the separation systems of the invention contains discrete beds of at least two HPLC stationary phases, and has an outlet at one end, which contains a frit. Mobile phase flows over the stationary phase towards the outlet. Two stationary phases are contained within the column to construct the mixed phase. A skilled artisan can typically make these columns. An empty fused silica micro-electrospray tip (that contains a sintered glass frit at a tapered outlet end) is packed with the stationary phases. First, a slurry of stationary phase 2 (such as a reversed phase resin) in a suitable solvent (such as methanol or isopropanol) is placed into an enclosed device that allows nitrogen pressure to be exerted on top of the slurry to force the slurry into the micro-electrospray tip (e.g., a PicoTip™, New Objectives Cambridge Mass., USA). The tip is monitored under a stereomicroscope, and once the desired length of stationary phase 2 has been packed, the nitrogen pressure is carefully released (so as not to cause a rapid change of pressure in the column which causes the packed bed to loosen and unpack). Subsequently, the slurry of stationary phase 2 is replaced with a slurry of stationary phase 1 (e.g., a SCX resin) in a suitable solvent (e.g., methanol) and nitrogen pressure is reapplied. Again the tip is monitored under a stereomicroscope to view the stationary phase so that nitrogen pressure can be released when the desired length of stationary phase 1 has been packed into the column. Desired lengths of each stationary phase are dependent upon the experiment for which the column is to be used. Typical lengths of stationary phase 2 are 2 to 6 cm and typical lengths of stationary phase 1 are 0.5 to 3 cm. A typical internal diameter of the micro-electrospray tip is 25 to 75 um. Once packed, these columns are washed with methanol and then a 50:50 v/v mixture of the mobile phases that are used for chromatographic separation of peptides (e.g., mixtures of acetonitrile and water modified with ammonium hydroxide). Further washing with one or two gradient s from water to acetonitrile modified with ammonium hydroxide and separation of a test peptide mixture (comprising 2 to 12 synthetic peptides) is performed before the column can be used for separation and analysis of peptide mixtures of interest.

### Example 12

### Decoupled SCX/micro-capillary reversed phase LC/MS/MS

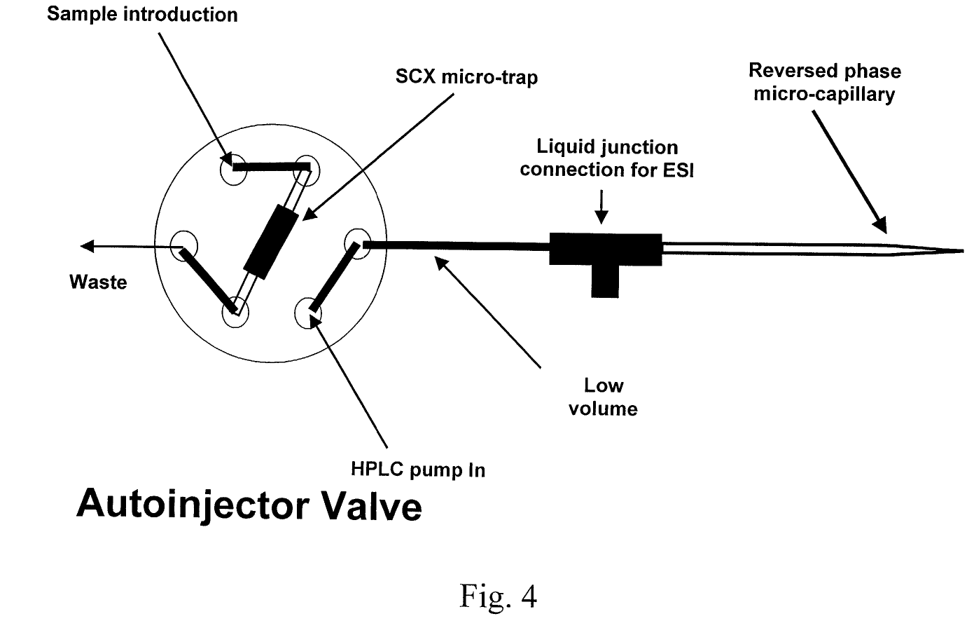

FIG. 4 depicts a decoupled SCX/micro-capillary reversed phase LC/MS/MS system. A microcapillary trap of SCX material was in place of the sample loop of the autosampler. Peptide mixtures were loaded in acidic solution, after which the SCX trap was switched into the flow stream of a high pH aqueous phase mobile. Increasing pH eluted peptides from the SCX phase, and a gradient delay of the reversed phase solvents ensures that the acetonitrile content of the mobile phases remains low to ensure that peptides of the mixture are refocused on the reversed phase material of the microcapillary analytical column. This approach promotes a high chromatographic performance of the automated microcapillary LC/MS/MS system, and enhanced the approach for sequencing peptides that were extremely hydrophilic. Furthermore, a high level of acetonitrile did not disrupt peptide adsorption on the SCX phase. Therefore, this approach enabled direct analysis of peptides that were fractionated by reversed phase chromatograph. This minimized sample manipulations and prevented analyte losses. For the decoupled SCX/reversed phase LC/MS/MS approach, high TFA concentration used to achieve highest peptide resolution for off-line reversed phase peptide fractionation was also advantageous, as this reagent promoted peptide adsorption on the SCX phase.

### Example 13

### Identifying and Quantifying Peptides

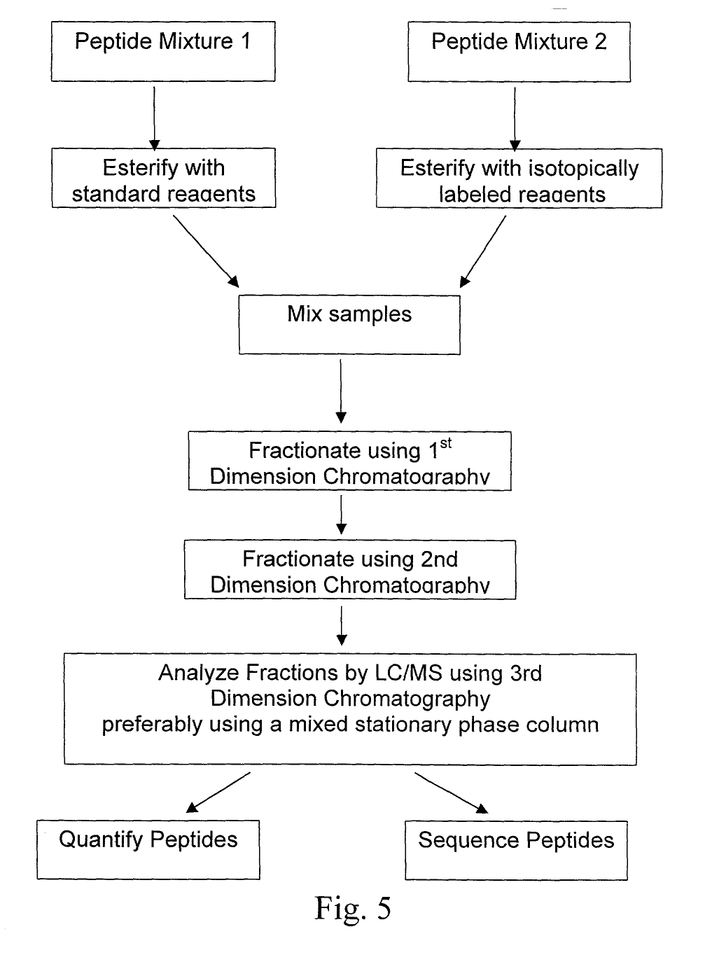

FIG. 5 depicts a flow chart demonstrating an example of a workflow used in quantifying and/or sequencing peptides as described herein. A first peptide mixture is esterified with standard reagents. A second peptide mixture is esterified with isotopically labeled reagents. The samples are mixed in their entirety. Following mixing, the sample is fractionated using a first dimension chromatography and then further fractionated using a second dimension chromatography. The fractions that are generated are analyzed by LC/MS using a third dimension chromatography, preferably a mixed stationary phase column. Peptide samples may either be quantified or sequenced from this analysis.

### Example 14

### Construction of a Kit for Use in Identifying and Quantifying Peptides

A kit may be used for quantifying and/or sequencing peptides as described herein. The kit can contain components that are used towards this end. Components of a kit can include: microchemistry reagents (e.g., reagents that permit the generation of peptide esters according to the methods of the invention); chromatography columns (e.g., 1 mm×150 mm stainless steel tube packed with a stationary phase that has containing frits at either end of the tubing); a standard peptide (e.g. a synthetic test peptide); a mobile phase (e.g., mixtures containing acetonitrile and water and modified with either an organic acid of an ammonium salt; typical examples include but are not limited to water/acetonitrile/trifluoro acetic acid in various compositions e.g., 95:5:0.1 v/v/v or 5:95:0.1 v/v/v; a mixed bed column (e.g., as described in Example 11)); and/or buffers to enable chromatography (e.g. water/acetonitrile/ammonium hydroxide in various compositions e.g., 95:5:0.1 v/v/v or 5:95:0.1 v/v/v). The kit may optionally include a set of instructions that instructs a user to use the components of the kit to carry out a method described herein, e.g., to effect the quantification or sequencing of a peptide or peptides.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for preparing a peptide ester, the method comprising:
providing an acidified alcohol solution;
providing a peptide sample comprising a peptide species; and
mixing the acidified alcohol solution and the peptide sample to form a mixture and thereby generate an ester of the peptide species, wherein concentration of the peptide species in the mixture is less than 1 nM.

2. The method of claim 1, wherein the alcohol is selected from the group consisting of methanol, ethanol, propanol, and isopropanol.

3. The method of claim 1, wherein the alcohol is a substituted alcohol selected from the group consisting of aminoethanol, trialkyl ammonium ethanol, biotinylated alcohol, and histidine labeled alcohol.

4. The method of claim 2, further comprising adsorption of the peptide species onto a solid phase prior to the mixing step.

5. The method of claim 4, wherein the solid phase comprises a hydrophilic chromatography phase.

6. The method of claim 4, wherein the solid phase comprises a strong cation exchanger.

7. The method of claim 1, wherein the ester is a methyl ester.

8. The method of claim 1, further comprising sequencing the peptide species after generation of the ester.

9. A method for preparing a peptide methyl ester, the method comprising:
providing a first solution comprising diazomethane and a solvent, wherein the solvent is miscible in water; and
mixing the first solution with a second solution, wherein the second solution is aqueous and comprises a peptide species, to thereby form a methyl ester of the peptide species.

10. The method of claim 9, wherein the solvent is selected from the group consisting of methanol, ethanol, isopropanol, acetonitrile, ethanolamine and triethanolamine.

11. The method of claim 9, further comprising sequencing the peptide species after formation of the methyl ester.

12. A method of determining the relative quantity of a peptide species in a mixture of peptides, the method comprising:
providing a first sample comprising a first population of the peptide species, wherein the concentration of the peptide species in the first sample is less than 1 nM;
esterifying the first population of the peptide species to form a first population of peptide esters;
providing a second sample comprising a second population of the peptide species, wherein the concentration of the peptide species in the second sample is less than 1 nM;
esterifying the second population of the peptide species with an isotopically enriched reagent to form a second population of isotopically labeled peptide esters;
mixing the first population of peptide esters with the second population of isotopically labeled peptide esters to form a mixture;
separating the mixture into a plurality of fractions;
analyzing a fraction with a mass spectrometer to obtain a first signal for the first population of peptide esters and a second signal for the second population of isotopically labeled peptide esters; and
determining the relative quantity of the peptide species in the first sample as compared to the second sample.

13. The method of claim 12, wherein the first population of peptide esters comprises peptide methyl esters.

14. The method of claim 12, wherein the second population of isotopically labeled peptide esters comprises peptide methyl esters.

15. The method of claim 12, wherein the second population of the peptide species is labeled with a stable isotope selected from the group consisting of deuterium, carbon-13, nitrogen-15 and oxygen-18.

16. The method of claim 12, wherein the second population of the peptide species is esterified using a solution comprising an alcohol.

17. The method of claim 12, wherein the second population of the peptide species is esterified using a solution comprising a substituted alcohol.

18. The method of claim 12, wherein the first sample and second sample comprise biological material derived from the same cell type or tissue type.

19. The method of claim 12, wherein the first sample and second sample comprise biological material derived from different cell types or tissue types.

20. The method of claim 12, wherein the determining step comprises ascertaining the ratio of hydrogen in the first population of peptide esters to deuterium in the second population of isotopically labeled peptide esters.

21. The method of claim 12, further comprising sequencing the peptide species after determining the relative quantity of the peptide species.

22. A method of fractionating peptides, comprising:
providing a sample comprising a plurality of different peptides;
adsorbing the peptides onto a strong cation exchanger;
selectively desorbing a first subset of the peptides by the action of a first mobile phase;
adsorbing the first subset of peptides onto a reversed phase HPLC column;
selectively eluting a second subset the peptides from the reversed phase HPLC column by a second mobile phase that develops an increasing acetonitrile concentration gradient; and
collecting peptide fractions eluted from the reversed phase HPLC column.

23. The method of claim 22, wherein the reversed phase HPLC column is a micro-HPLC column comprising both a reversed phase and a strong cation exchanger stationary phase, and wherein the second mobile phase comprises both an acetonitrile gradient and a pH gradient.

24. The method of claim 23, further comprising sequencing a peptide contained in a peptide fraction by mass spectrometry.

25. A peptide separation system, comprising:
a column comprising an ion exchange stationary phase and a reversed phase HPLC phase, wherein the ion exchange stationary phase has strong cation exchange characteristics, and wherein the reversed phase HPLC has C2, C4, C8, C18 or polymeric characteristics; and
a mobile phase gradient comprising an acetonitrile gradient and a pH gradient.
